(19) United States
(12) Patent Application Publication
(10) Pub. No.: US 2011/0189759 A1
(43) Pub. Date: Aug. 4, 2011

(54) METHOD AND DEVICE FOR THE DIGETSION OF BIOLOGICAL CELLS

(76) Inventors: Ralf Himmelrich, Langenfeld (DE); Thomas Rothmann, Langenfeld (DE)

(21) Appl. No.: 12/863,523

(22) PCT Filed: Jan. 15, 2009
(86) PCT No.: PCT/EP09/50440

§ 371 Date: Sep. 8, 2010

(30) Foreign Application Priority Data
Jan. 24, 2008 (EP) 08150595.0

(51) Int. Cl. C12N 1/06; C12M 1/33

(52) U.S. Cl. 435/259; 435/306.1

(57) ABSTRACT

The invention relates to a method and a device for the digestion of biological cells. Digestion of cells is understood as breaking up cells so that hereditary information can be removed. Using the present invention, nucleic acids are to be able to be removed from cells in particular. The nucleic acids include RNA and DNA. According to the invention, cells are digested in a vessel which can be connected to a line and can thus be part of an overall device. Liquid can drain out of the container or can be pumped into the container via the line, for example. The typical means for performing a mechanical digestion are located in the vessel, that is, in particular beads and a buffer solution. In contrast to the prior art, however, the vessel is not shaken, that is, subjected to vibrations. Instead, it is stirred with the aid of a magnetic stirrer. Strong vibrations are thus avoided. A vessel for performing digestion of cells can therefore be integrated without problems in an overall device.

The invention relates to a method and device for disrupting biological cells, which is known, for example, from the documents “Huh Yun Suk et al, Microfluidic cell disruption system employing a magnetically actuated diaphragm, Electrophoresis December 2007, vol. 28, No. 24, pages 4748-4757” and “Haugland et al., Evaluation of different methods for the extraction of DNA from fungal coninia by quantitative competitive PCR analysis, Journal of Microbiological Methods, Elsevier, Amsterdam, NL, vol. 37, 1. January 1999, pages 165-176. The disruption of cells is understood to mean breaking up the cells in order to be able to extract genetic information. The present invention is supposed to enable the extraction of nucleic acids from cells in particular. These include RNA and DNA. Furthermore, proteins are released from the interior of the cells with the present invention.

Cells can be disrupted with varying difficulty. Cultivated cell cultures can generally be disrupted easily with means that conserve genetic information. In contrast, spores are comparatively difficult to disrupt. The means required for disrupting such cells not only break up the cells, but also fragment genetic information comprehensively.

White blood cells are an example for cells that can be easily disrupted. They can be disrupted comparatively gently, for example by means of the enzyme proteinase K in the presence of a detergent (e.g. sodium dodecyl sulfate or Triton X-100). Nucleic acids are sheared in every cell disruption and fragmented by double-strand breaks in the genetic material. However, the genetic information is not fragmented to such a degree that the desired analyses would be jeopardized thereby.

The larger the extent of fragmentation of nucleic acids is, the more problematic it is to perform the desired analyses. Therefore, reducing the double-strand breaks during the disruption of cells to a tolerable extent is one goal.

If cells are to be disrupted that are difficult to disrupt, this is done, for example, by supplying heat. In that case, cells that are difficult to disrupt are treated, for example, for 10 minutes at 95° C. Many bacteria are typically disrupted by means of heat.

Disruption by means of ultrasound is another method used in the case of cells that are difficult to disrupt. However, ultrasound fragments genetic information particularly comprehensively. Therefore, ultrasound is commonly used only in cases where other methods fail. For example, spores, which are particularly resistant, are disrupted by means of ultrasound.

Milling by means of glass beads constitutes a third method for disrupting cells that are difficult to disrupt. Bacteria and fungi are disrupted by means of glass beads. Disruption using glass beads (also known as “bead beating” among experts) proceeds comparatively gently in comparison to ultrasound and heat. However, the genetic information is fragmented in varying extents in all three cases.

If glass beads are to be used for disrupting, then a vibrating or shaking machine is generally used, such as a so-called Vortex® vibrator. One or more tubes of plastic are filled with an aqueous solution comprising a lysis buffer, with the cell material and glass beads, sealed and put into the vibrating or shaking device and shaken for five minutes, for example, by means of the device. Such a method is also called “vortexing with glass beads” or “bead beating”.

If the cell disruption is completed, the tube or tubes are removed, the tubes are centrifuged, if required, in order to separate glass particles from the liquid, and the liquid contents are put into another tube. Here, the disrupted cell material is treated with other buffer solutions in order to process the nucleic acids.

Disruption using glass beads is described, for example, in the document DE 10 2004 032 888 B4.

Though this method is an established process which works reliably it can, however, hardly be automated, especially not if automation should or must require only little space. The strong vibrations, which can cause damage if tubes serving as lysis vessels are part of the entire device, are problematic.

A method and device for lysing microorganisms is known from document U.S. Pat. No. 6,632,662 B1. Beads consisting of glass having a diameter of 50 μm and one or more substantially larger magnetic beads having a diameter of, for example, 2 mm, are located in a container. The beads with the large diameter are rolled along a runway or outer wall of a vessel using a magnet. Together with the beads of glass which are located between the runway and the large beads, the beads crush biological material. Accordingly, document U.S. Pat. No. 6,632,662 B1 teaches to press beads together and against a wall, similar to millstones of a mill, and thus use them for crushing. If a single larger bead is rolled along a vessel wall at high speed, the vessel concerned is disadvantageously made to oscillate and thus vibrates. Though this could be compensated by at least one other bead also rolling at the same speed on the opposite side in a suitable way, however, this would require an exact control of the large magnetic beads, which on the one hand is not known from U.S. Pat. No. 6,632,662 B1, and which would also, on the other hand, be relatively complex. Another drawback is that the beads do not act in space and that they do not contribute to crushing biological material especially above the beads, so that the beads are relatively inefficient.

The object of the invention is providing a method and a device for the efficient disruption of cells. Preferably, vibrations of a vessel in which the disruption is being carried out are to be avoided during disruption.

In order to achieve the object, cells are disrupted in a vessel which is connected to a pipe and which can thus be part of an overall device. Liquid can drain off from the vessel via the pipe, or be pumped into the vessel. The known means for carrying out a mechanical disruption, that is, in particular beads and a buffer solution, are located in the vessel. As an alternative to glass beads, beads consisting of zirconium or silicon carbide particles in a suitable size can also be used. In contrast to the prior art, however, the vessel is not shaken, i.e. not subjected to vibrations. Stirring is carried out instead, using a stirring member, namely in particular using a magnetic stirrer preferably comprising a stirring rod located in the vessel. Strong vibrations are thus avoided in principle. A vessel for carrying out a disruption of cells can therefore be integrated into an overall device or overall system without any problems.

Vibrating a vessel, which is for example connected to a line, in a manner known from the prior art, i.e. exposing it to vibrations, is problematic. This especially applies if miniaturization is a goal. However, the experts were of the opinion that vibratory movements generating constantly changing directions of movement of the glass beads were required in order to be able to disrupt cells even of bacteria and fungi in an efficient manner.

Vibratory movements of the glass beads as they can be generated using vibrating devices cannot be imitated, even approximately, by means of a magnetic stirrer or another stirring tool. According to the prior art, a magnetic stirrer therefore serves for mixing contents in a vessel, but not as a means for bringing about a mechanical disruption of cells. Therefore, moving particles or beads for mechanically disrupting cells, using, in particular, a magnetic stirrer or stirring tools with the same effect, is not known from the prior art.

Surprisingly, it was found that the mechanical disruption caused with a stirring rod of a magnetic stirrer is just as efficient as the mechanical disruption with a vibrating device known from the prior art. It was investigated whether a frequent change of the direction of rotation of the magnetic stirrer was required for an efficient disruption in order thus to imitate a vibratory movement. Unexpectedly, it was found that changing directions of rotation of the magnetic stirrer only have little influence on efficiency. Thus, it is already sufficient to provide only one direction of rotation of the magnetic stirrer in order to be able to disrupt efficiently.

The use of the magnetic stirrer achieves that the vessel is not vibrated. Therefore, integrating the vessel into a mechanically sensitive overall device now is not problematic. Otherwise, as tests have shown, damage is very easily generated by vibrating, for example leaking transitions between pipes. Moreover, sensitive parts of an overall device for automatic or semi-automatic processing can easily be damaged by vibrating.

In one embodiment, the magnetic stirrer comprises a magnetic stirring rod. In an advantageous embodiment of the invention, the length of the stirring rod is selected such that it cannot lie horizontally on the bottom of the vessel. In the idle state, the stirring rod as a rule then rests on the bottom of the vessel—which can be a vessel bottom, a filter or a frit—with one end, and is adjacent to a vessel wall with the other end. Thus, depending on the embodiment, the length of the stirring rod is longer than the diameter of the bottom of the vessel, or of the diameter of a frit mounted horizontally in the vessel positioned in accordance with its intended use. It was found that, as a rule, the efficiency of the disruption can be significantly improved by this embodiment. Though a slight imbalance occurs, as a rule, it only causes slight vibration, however. In principle, an integration of the vessel into a sensitive overall device is still possible. However, should the imbalance prove to be too great, it is sufficient to select the length of the stirring rod to be slightly shorter in order thus to sufficiently reduce the inclination and thus, the imbalance which occurs. Therefore, no large technical effort has to be taken to keep vibration sufficiently small.

However, if the liquid volume is very small, it may be sufficient to insert a stirring rod of a magnetic stirrer so as to lie horizontally, without having to accept noticeable limitations with regard to the efficiency of the method. In this embodiment, any imbalance is avoided so that vibrations are avoided particularly well.

In one embodiment of the invention, the vessel comprises a filter or a frit at the bottom. The filter or the frit serves for filtering out particles or beads when the liquid content is drawn out via a pipe, for example. Thus, centrifuging in order to separate the glass beads from the liquid, which is known from the prior art, can be dispensed with. Moreover, a pipe is prevented from becoming clogged by the particles or beads.

Typically, the diameter of the particles or beads, which preferably consist of glass, is approximately 600 μm. Therefore, the diameter of the pores of the filter or frit preferably is up to 100 μm, in particular 50 to 100 μm, in order to separate the liquid and the cell material in it reliably from the particles.

A frit consisting of polypropylene is particularly suitable as a filter.

In order to be able to disrupt particularly efficiently, the level of the liquid in the vessel should be low. Though this can be attained with a large base surface area of the vessel, it is difficult in this case to draw out the liquid content as completely as possible, for example via a pipe mounted at the bottom. Thus, a conical shape of the vessel has proved advantageous, with the diameter of the vessel increasing in the upward direction. Gently inclined or plane surfaces in the vessel are thus advantageously avoided or minimized. The base surface area of the vessel can be small without having to accept a high level of liquid in the vessel.

Preferably, as is known from the prior art, the vessel is sealed by a corresponding cap during disruption.

It is possible in principle to provide a different mixing tool for stirring instead of the magnetic stirrer. However, a magnetic stirrer has proven particularly suitable. Because in that case, no shaft has to extend into the vessel, for example through a cap, which can cause unwanted vibrations that are avoided by using a magnetic stirrer. Furthermore, a magnetic stirrer works without contact as regards actuation. The vessel is sealed during the process, so that no sample material can escape. This is advantageous in particular if potentially infectious sample material is concerned.

A stirring member used for stirring is able to execute different stirring movements. In particular, circular stirring processes are executed. Preferably, a stirring member located in the vessel is driven contactlessly. The stirring member can have different lengths relative to the diameter of the vessel. The stirring member can have different geometries and preferably has the geometry of a rod. The vessel and, in particular, the inner wall of the vessel can be configured in different ways. Preferably, it has a smooth inner wall and it is a conical vessel. A solution located in the vessel can additionally contain enzymes for cell disruption.

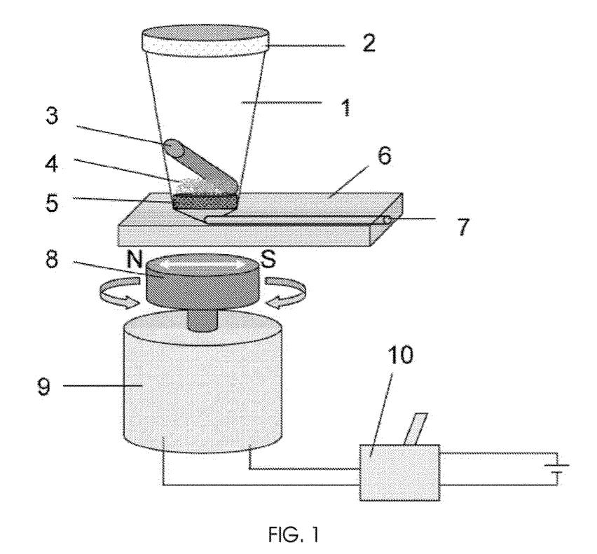

FIG. 1 schematically shows the structure of the device for an automated processing of biological samples. A conical vessel 1 is sealed with a cap 2 in order to prevent an unwanted escape of liquid and other material from the vessel—which is hereinafter referred to as a lysis chamber. In the vessel 1 (bottom diameter 0.7 cm; top diameter 2.0 cm; inner height 2.5 cm; volume approx. 3.2 ml), there is a magnetic stirring rod 3 used for stirring during the disruption of cells. Moreover, the vessel 1 contains glass beads 4 resting on a frit 5 consisting of polypropylene. The diameter of the frit is 7 mm. The frit 5 is 1.5 mm thick and provided with penetrating pores having a diameter of 50 to 100 μm. Since the magnetic rod 3 is longer than the diameter of the frit 5, it cannot rest horizontally on the frit forming the bottom of the vessel. The vessel 1 is placed on a substrate 6 which is provided with a funnel-shaped depression underneath the frit. The bottom end of the funnel-shaped depression opens into a channel 7. A magnet 8 is mounted, horizontally aligned, on the axis of the electric motor, underneath the substrate 6 and the vessel 1. The direction of rotation of the electric motor can be changed with a polarity-reversing switch 10.

The apparatus shown in FIG. 1 is part of a microfluidic system which comprises further devices or is connected to devices in order to be able to carry out processing automatically or semi-automatically. This includes, in particular, a pump with which a lysis buffer solution can be pumped off. In one embodiment, the system also comprises a vessel with a lysis buffer solution contained therein in order to be able to pump the buffer solution into the vessel 1.

The inner diameter of the channels of the microfluidic system is no larger than 1 mm, in particular the inner diameter of the channel 7 shown in FIG. 1.

Preferably, both the substrate 6 on which the vessel 1 is placed and the vessel 1 are parts that are or can be produced by injection-molding in order to enable an inexpensive fabrication. These parts are suitable assembled so that an opening at the bottom of the vessel 1 is connected with an opening of a microchannel 7. The parts 1 and 6 are then welded to each other, preferably by heat, so that a reliably tight connection is produced between the channel 7 and the vessel 1. Typically, the material of the vessel 1 is polypropylene. Polycarbonate or cyclic olefin copolymers (COC) are the preferred material for the substrate. Polycarbonate and COC are transparent, hard and dimensionally stable. They thus meet the requirements for a substrate in automatic processes, in particular with regard to dimensional stability. The person skilled in the art may also use alternative polymers with the same requirements for injection-molding. The requirements with regard to the dimensional stability of the lysis vessel 1 are lower. Thus, polypropylene is sufficient as the material for the vessel 1.

As a rule, the vessel 1 is exposed to a maximum temperature of 80° C. In contrast, the substrate 6 can be exposed to temperatures of up to 100° C. within the context of automatic processing. For this reason, a relatively thermally stable material is preferred as the substrate material.

The channel 7 to which the vessel 1 is connected can be opened and closed by means of a corresponding valve. The channel 7 is closed during the disruption of cells. For disruption, the sample containing the cells is filled into the vessel, for example in a known manner, as well as a lysis buffer and glass beads.

In order to prove the efficiency of the device according to the invention or of the method according to the invention, various samples were processed both according to the prior art as well as by means of the invention, as will be described below.

Results obtained with the invention will be illustrated, also referring to comparative tests, below.

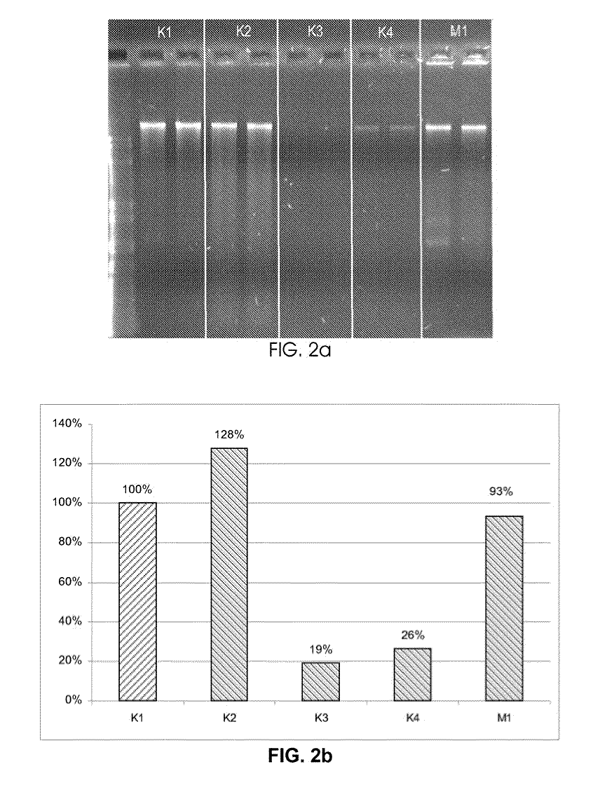

The first test series relates to the nucleic acid preparation of Corynebacterium glutamicum. The results obtained will be shown in FIGS. 2a and 2b.

On the one hand, preparations were carried out in accordance with the following protocol for nucleic acid preparation from liquid cultures of microorganisms, which corresponds to the prior art.
1. Aliquot 250 μl bacteria or yeast liquid culture in 1.5 ml microcentrifuging tubes
2. Centrifuge for 10 min at 5000×g and discard supernatant
3. Re-suspend cell sediment in 600 μl lysis buffer [differentiate between bacteria lysis buffer and fungi lysis buffer; in the case of yeast, optionally use 20 μl zymolase]
4. Add 200 mg glass beads (glass beads with a diameter of 600 μm) [Sigma-Aldrich Art. No. G8772-500G]
5. Mix for 5 min at maximum power on a vortex mixer with a disc attachment for 1.5 ml microcentrifuging tubes
6. Add 600 μl buffer AL [QIAGEN; QIAamp DNA Micro Kit]
7. Add 20 μl proteinase K [QIAGEN; QIAamp DNA Micro Kit]
8. Mix and incubate for 10 min at 56° C.
9. Short centrifuging for 20 sec.
10. Transfer the entire supernatant into a new 1.5 ml microcentrifuging tube
11. Add 600 μl ethanol
12. Pour mixture to QIAamp MinElute Spin Column (several times)
13. *Binding: centrifuge for 1 min at 6000×g
14. *Washing steps: 500 μl buffer AW1, 1 min at 6000×g; 500 μl buffer AW2, 3 min at max. g
15. *Dry-spin for 1 min at maximum g
16. For elution of the nucleic acids, elute with 80 μl buffer TE, 1 min at 6000×g

*) Steps according to the “QIAamp DNA Micro Handbook”, which can be obtained via the internet, inter alia, at http://www1.qiagen.com/HB/QIAampDNAMicroKit_EN

Lysis Buffer:
Lysis buffer for bacteria, e.g. Corynebacterium glutamicum 1.2% Triton with 20 mg/ml lysozyme
Add 10 μl Reagent DX (silicone antifoaming emulsion, Wacker Chemie AG) to 6 ml lysis buffer
Lysis buffer for fungi, e.g. Saccharomyces cerevisiae 1M Sorbitol; 100 mM EDTA
Optionally, 20 μl zymolase (2.5 mg/ml)/sample additionally

Moreover, according to the invention, nucleic acid was processed in accordance with the following protocol (protocol within the sense of the invention):
1. Aliquot 250 μl bacteria or yeast liquid culture in 1.5 ml microcentrifuging tubes
2. Centrifuge for 10 min at 5000×g and discard supernatant
3. Re-suspend cell sediment in 600 μl lysis buffer and transfer to lysis chamber (vessel 1, as shown in FIG. 1) [differentiate between bacteria lysis buffer and fungi lysis buffer; in the case of yeast, optionally use 20 μl zymolase]
4. Add 200 mg glass beads, diameter 600 μm [Sigma-Aldrich Art. No. G8772-500G] to the lysis chamber
5. 10 min magnetic stirring (centrically) at the highest possible number of revolutions without the lysate being spattered; change stirring direction every 20 sec by reversing polarity of the electric motor
6. Add 600 μl Puffer AL [QIAGEN; QIAamp DNA Micro Kit], transfer with tip cut open into 1.5 ml microcentrifuging tubes
7. Add 20 μl proteinase K [QIAGEN; QIAamp DNA Micro Kit]
8. Mix and incubate for 10 min at 56° C.
9. Short centrifuging for 20 sec.
10. Transfer the entire supernatant into a new 1.5 ml microcentrifuging tube
11. Add 600 μl ethanol
12. Pour mixture to QIAamp MinElute Spin Column (several times)
13. *Binding: centrifuge for 1 min at 6000×g
14. *Washing steps: 500 μl buffer AW1, 1 min at 6000×g; 500 μl buffer

AW2, 3 min at max. g
15. *Dry-spin for 1 min at maximum g
16. *For elution of the nucleic acids, elute with 80 μl buffer TE, 1 min at 6000×g

After preparation in accordance with the prescribed protocols, the nucleic acids were electrophoretically separated on an agarose gel. After separation, the nucleic acid fragments were colored in an ethidium bromide-containing water bath, caused to light up on a UV light panel and photographed by means of a photodocumentation system. The genomic DNA can be discerned as a bright band in the upper third of the FIG. 2a.

FIG. 2a relates to the nucleic acid preparation of Corynebacterium glutamicum, 0.7% agarose gel (application 15 μl eluate). In the process, a solution of 0.7% w/v agarose is heated until the agarose particles melt, and after homogenization, they are left to rigidify in a gelling form. The gel contains pockets into which the nucleic acid-containing solution was pipetted and electrophoretically separated. In this case, 15 μl of the eluate was “applied” after nucleic acid preparation, with

K1: Reference protocol with vortex mixer without lysozyme in the lysis buffer

K2: Reference protocol with vortex mixer with lysozyme in the lysis buffer

K3: Reference protocol without vortex mixing and without lysozyme in the lysis buffer

K4: Reference protocol without vortex mixing but with lysozyme in the lysis buffer

M1: Protocol according to the invention without lysozyme in the lysis buffer.

The strength, in other words, the light intensity (number and surface area of white pixels) of a band in FIG. 1A is a measure for the quality of the cell disruption. The light band at the boundary of ⅓ to ⅔ of the vertical of the image is of primary importance.

The bands K1 and K2, or rather, the bands resulting from the tests K1 and K2, are light and broad compared with the bands K3 and K4, i.e. the bands obtained in the context of the tests K3 and K4. The bands K3 and K4, in contrast to the bands K1 and K2, were obtained without vortex mixing. This illustrates that a mechanical disruption is indispensable for disrupting with useful results. The bands K1 and K2 correspond to the band M1 obtained by the method according to the invention with the device shown in FIG. 1. It follows that the mechanical disruption according to the present invention does not exhibit any difference with regard to quality to the mechanical disruption according to the prior art.

In order to further illustrate that the mechanical disruption is crucial with regard to quality, comparative tests were performed with and without lysozyme. Lysozyme is a hydrolase which attacks the cell wall of bacteria. The comparison of the band K3 (lysozyme was not used) with K4 (lysozyme was used) shows a discernible influence in the case of the disruption, which is of little use otherwise, because the band K4 is still faintly perceptible in the illustration, in contrast to the band K3. However, if disruption is performed mechanically, and thus in a useful manner, then no influence can be recognized anymore in FIG. 2a, as the comparison of the band K1 (lysozyme was not used) with the band K2 (lysozyme was used) shows. Thus, the use of lysozyme does not become noticeable with respect to quality.

Another band is visible directly “at the gel pocket” above the band M1, i.e. the band that was obtained within the context of the test performed according to the invention. This means that the disruption method, compared to vortex mixing in accordance with the prior art, proceeds more gently, that is, there is less shearing. In this regard, the method according to the invention is even superior to the prior art.

FIG. 2b also relates to the aforementioned nucleic acid preparation of Corynebacterium glutamicum. What is shown are the measured optical densities (OD) relative to the optical density obtained in the test K1. The optical density was in each case determined with a wavelength of light of 260 nm. The optical density is a measure for the efficiency of the cell disruption as regards quantity. The comparison of the optical densities in the case of the tests K1 and K2 illustrates that the use of lysozyme has a positive effect as regards quantity, because the optical density of the test K2, at 128%, was significantly higher as compared with the optical density of the test K1, in which a disruption was also performed mechanically, in accordance with the prior art, but in which no lysozyme was used, in contrast to the test K2. The disruption M1 performed according to the invention, in which no lysozyme was used, led to an optical density, which at 93% comes up to the optical density of the test K1. Thus, a result in the same order of magnitude was thus obtained as regards quantity.

The tests K3 and K4, which were performed without mechanical disruption, in contrast led to lower optical densities of only 19% in the case where there was no lysozyme, or 26% in the case where lysozyme was used, in comparison to the density obtained with the test K1. Therefore, mechanical disruption is crucial for obtaining a quantitatively good result. Though a noticeable improvement can also be obtained in the case of a mechanical disruption by the additional use of lysozyme, however, mechanical disruption remains pivotal also in a quantitative respect. If disruption is performed in accordance with the invention, then a very good result is also obtained which approximates the mechanical disruption in accordance with the prior art.

Thus, bacteria, which are problematic to disrupt, can be successfully disrupted by means of the invention.

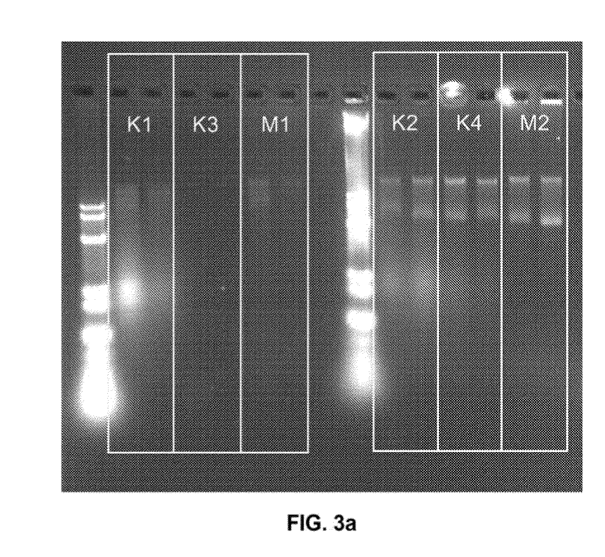

The second test series that was carried out relates to the nucleic acid preparation of Saccharomyces cerevisiae (yeast), 0.7% agarose gel (application 15 μl eluate). The above-mentioned protocols were applied. The results obtained will be shown in FIGS. 3a and 3b.

The following tests were performed:

K1: Reference protocol with vortex mixer without zymolase in the lysis buffer

K2: Reference protocol with vortex mixer with zymolase in the lysis buffer

K3: Reference protocol without vortex mixer and without zymolase in the lysis buffer

K4: Reference protocol without vortex mixer but with zymolase in the lysis buffer

M1: Protocol according to the invention without zymolase in the lysis buffer.

M2: Protocol according to the invention with zymolase in the lysis buffer.

Corresponding to FIG. 2a, FIG. 3a shows the qualitatively obtained result of the nucleic acid preparation of Saccharomyces cerevisiae, 0.7% agarose gel (application 15 μl eluate).

FIG. 3b relates to the nucleic acid preparation of Saccharomyces cerevisiae. The results of the quantitative real time PCR for the detection of genomic Saccharomyces cerevisiae are shown. The so-called “threshold cycle” (Ct) is shown.

Polymerase chain reaction (PCR) is a method for amplifying DNA in vitro, i.e. without using a living organism, such as the bacterium Escherichia coli or baker's yeast Saccharomyces cerevisiae. Real time-PCR (abbreviated RTD-PCR) is an amplification method for nucleic acids based on the principle of conventional polymerase chain reaction (PCR), and which additionally offers the possibility of quantification. Quantification is carried out by means of fluorescence measurements at the end or during a PCR cycle (hence the name “real time”) and thus differs from other quantitative PCR methods (qPCR) which are only quantitatively evaluated after PCR is complete (e.g. competitive PCR). Fluorescence increases proportionally to the amount of the PCR products, which makes quantification possible. A gel-electrophoretic separation of the fragments is not necessary, the data are immediately available and the risk of contamination is low.

In the first phase of amplification of a PCR, the template quantity is limited and the probability of an encounter of template, primer and polymerase is suboptimal, whereas, in the third phase of the amplification, the quantity of the products (DNA, pyrophosphate, monophosphate nucleotides) increases in such a degree that an inhibition occurs caused by them, product fragments hybridize with each other frequently, the substrates are slowly used up and finally, the polymerases and nucleotides are slowly destroyed by the heat. An exponential and thus quantifiable increase can only be found in the intermediate phase. Exponentially, a PCR remains at 12 to 400 starting copies for approx. 30 cycles, at 200 to 3200 for 25 cycles, and at initially 3200 to 51200 for a maximum of 20 cycles. In order always to be able to perform measurements at the beginning of an exponential phase, the CT value (threshold cycle) or the Cp value (crossing point), respectively, are used, which describes the cycle in which the fluorescence rises significantly above the background fluorescence for the first time.

FIG. 2a illustrates that, using the method according to the invention, the qualitatively best result was obtained also in the case of yeast, i.e. in the case of a fungus, because in the case of M2, another band is clearly visible in the area of the pockets in contrast to the other tests. In the case of yeast, however, the use of the enzyme zymolase has a significant effect upon the quality of the test results, as the comparison of the cases K1 K3, M1 with the cases K2, K4 and M2 shows. In the first three cases, the enzyme was not used, as opposed to the last three cases. In a qualitative respect, the influence due to the mechanical mixing is comparatively small, as the comparison between K2 and K4 shows in particular.

FIG. 3b shows a measure for the results in a quantitative respect. The smaller the value ct, the better the quantitative yield. The exponential relationship of the values must be taken into account with regard to the PCR. Thus, the difference of the values K3−K1=26.7−20.7=6 Cts. This means that the yield in the case K3 is lower by the factor 26=64. A Delta-Ct of approximately 0.4-0.5 is not significant, because such a value lies within the variance. If the Delta-Ct is 1, such as in the comparison of M1 with K1, the yield is 50% (less).

In a quantitative respect, the best results were obtained with the test M2, i.e. a method performed in accordance with the invention, in which zymolase was used. The comparison of the cases K2 with K4 and K1 with K3 illustrates that a mechanical disruption has considerable influence on the quantitative result.

This test series illustrates that, in the case of a yeast fungus, the method according to the invention is in any case superior to the prior art both in a qualitative and quantitative respect, if an enzyme that is suitable for disruption is used in addition to the mechanical influence.

The effect of a change of the direction of rotation of a magnetic stirrer on the test results was investigated in the context of a third test series.

The stirring direction can be made unidirectional or bidirectional by reversing the polarity of the electric motor. The protocol was executed in accordance with the first test series. What was investigated was constant stirring as well as bidirectional stirring by polarity reversal (the direction of rotation was changed by polarity reversal every 20 seconds during the ten-minute disruption), both with and without glass particles.

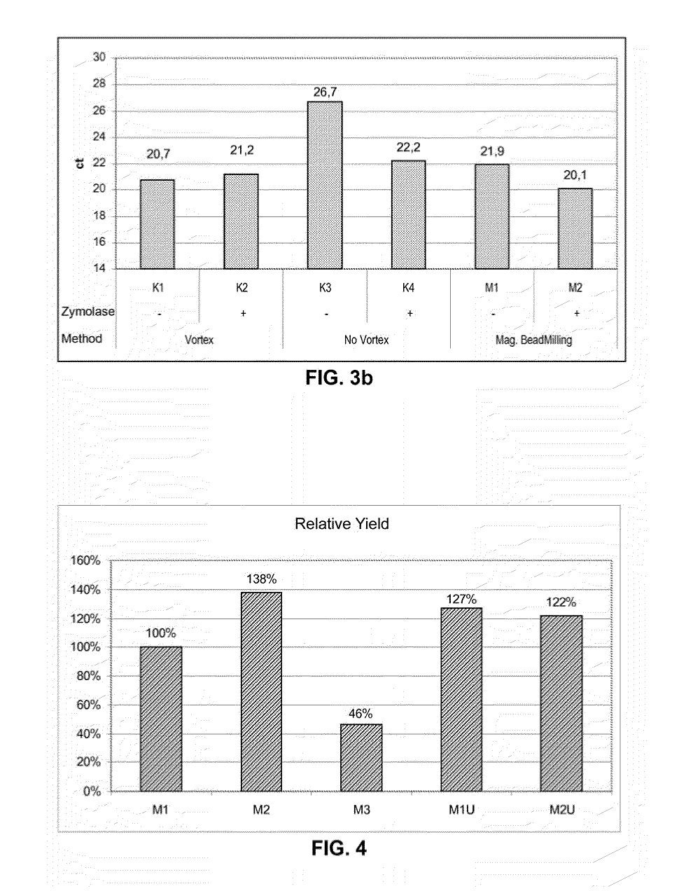

The quantitatively obtained ct results, which result from a performed real time PCR for the detection of genomic Corynebacterium glutamicum, are shown relatively in FIG. 4. The relative yield, normalized to the value “M1” is depicted.

The images in FIG. 4 relate to the following tests.

M1: Protocol according to the invention without polarity reversal of the stirring direction and without lysozyme in the lysis buffer.

M2: Protocol according to the invention without polarity reversal of the stirring direction and with lysozyme in the lysis buffer.

M3: Protocol according to the invention without polarity reversal of the stirring direction and without glass particles (glass beads) and without lysozyme in the lysis buffer

M1U: Protocol according to the invention with polarity reversal of the stirring direction and without lysozyme in the lysis buffer.

M2U: Protocol according to the invention with polarity reversal of the stirring direction and with lysozyme in the lysis buffer.

A comparison of the results of M1 with M1U and M2 with M2U, respectively, shows that a polarity reversal of the stirring direction is not required in order to attain good yields. Though, judging from the measured values, an improvement of the yield resulted from the polarity reversal in the case M1/M2U, however, the result was reversed in the case M2/M2U.

Moreover, the comparison M3 with M1 shows that the presence of particles is very important for improving the yield. Thus, a satisfactory disruption is not possible with a stirring tool alone.

A nucleic acid preparation of microorganisms from blood was analyzed in the context of a fourth test series.

Apart from the isolation of nucleic acids from liquid cultures of microorganisms that are difficult to lyse, the vortex reference protocol is also used in the analysis of patients' blood with suspected sepsis. For this reason, Corynebacterium glutamicum was added to human blood (stored blood) in this experiment. The nucleic acids were isolated using different protocols and detected by means of quantitative PCR.

Protocol in accordance with the prior art:
1. Aliquot 200 μl human blood and 20 μl Corynebacterium glutamicum overnight culture in 1.5 ml microcentrifuging tubes
2. Add to 380 μl lysis buffer+/−lysozyme
3. Add 200 mg glass beads 600 μm [Sigma-Aldrich Art. No. G8772-500G]
4. Mix for 5 min at maximum power on the vortex mixer with a disc attachment for 1.5 ml microcentrifuging tubes
5. Add 600 μl buffer AL [QIAGEN; QIAamp DNA Micro Kit]
6. Add 20 μl proteinase K [QIAGEN; QIAamp DNA Micro Kit]
7. Mix and incubate for 10 min at 56° C.
8. Short centrifuging for 20 sec.
9. Transfer the entire supernatant into a new 1.5 ml microcentrifuging tube
10. Add 600 μl ethanol
11. Pour mixture to QIAamp MinElute Spin Column (several times)
12. *Binding: centrifuge for 1 min at 6000×g
13. *Washing steps: 500 μl buffer AW1, 1 min at 6000×g; 500 μl buffer AW2, 3 min at max. g
14. *Dry-spin for 1 min at maximum g
15. *For elution of the nucleic acids, elute with 80 μl buffer TE, 1 min at 6000×g

*) Steps according to the “QIAamp DNA Micro Handbook”

### Lysis Buffer

Lysis buffer for bacteria, e.g. Corynebacterium glutamicum 1.2% Triton with 20 mg/ml lysozyme
Add 10 μl Reagent DX (silicone antifoaming emulsion, Wacker Chemie AG) to 6 ml lysis buffer

Protocol according to the invention
1. Aliquot 200 μl human blood and 20 μl Corynebacterium glutamicum overnight culture in 1 and in lysis chamber (see FIG. 1)
2. Add to 380 μl lysis buffer+/−lysozyme
3. Add 200 mg glass beads 600 μm [Sigma-Aldrich Art. No. G8772-500G]
4. 10 min magnetic stirring (centrically) at the highest possible number of revolutions without the lysate being spattered; stirring direction constant
5. Add 600 μl buffer AL [QIAGEN; QIAamp DNA Micro Kit] Transfer with tip cut open into 1.5 ml microcentrifuging tubes
6. Add 20 μl proteinase K [QIAGEN; QIAamp DNA Micro Kit]
7. Mix and incubate for 10 min at 56° C.
8. Short centrifuging for 20 sec.
9. Transfer the entire supernatant into a new 1.5 ml microcentrifuging tube
10. Add 600 μl ethanol
11. Pour mixture to QIAamp MinElute Spin Column (several times)
12. *Binding: centrifuge for 1 min at 6000×g
13. *Washing steps: 500 μl buffer AW1, 1 min at 6000×g; 500 μl buffer AW2, 3 min at max. g
14. *Dry-spin for 1 min at maximum g
15. *For elution of the nucleic acids, elute with 80 μl buffer TE, 1 min at 6000×g

The following tests were performed.

K1: Reference protocol with vortex mixer without lysozyme in the lysis buffer

K2: Reference protocol with vortex mixer with lysozyme in the lysis buffer

K3: Reference protocol without vortex mixing and without lysozyme in the lysis buffer

K4: Reference protocol without vortex mixing but with lysozyme in the lysis buffer

M1: Protocol according to the invention without lysozyme in the lysis buffer.

M2: Protocol according to the invention with lysozyme in the lysis buffer.

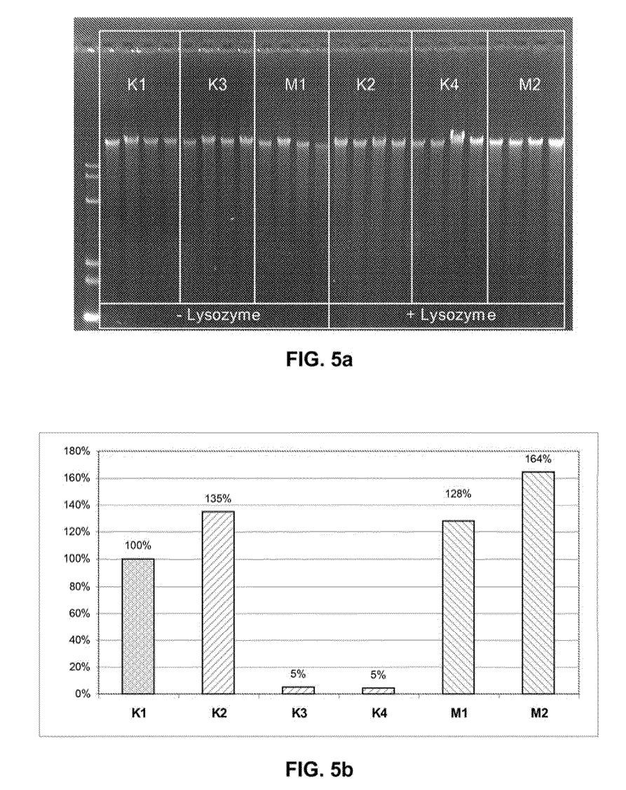

FIG. 5a illustrates the qualitatively obtained results of a nucleic acid preparation of a mixture of human blood and Corynebacterium glutamicum—agarose gel of the entire nucleic acid.

FIG. 5b shows the quantitatively obtained result of a nucleic acid preparation of a mixture of human blood and Corynebacterium glutamicum—results of the quantitative PCR for the detection of genomic Corynebacterium glutamicum DNA; relative yields normalized to the reference protocol K1.

As is clear from FIGS. 5a and 5b, the best result, on the whole, could be obtained by using the invention, namely in accordance with test M2. The lysis efficiency in the case of Corynebacterium glutamicum could be improved significantly by using lysozyme.

1. A method for disrupting cells by putting cells into a vessel together with beads and/or particles and a solution wherein the disruption is brought about by stirring with an optionally magnetic stirring member.

2. A method according to claim 1, wherein cells of bacteria or fungi are disrupted.

3. A method according to claim 1, wherein a stirring rod of the stirring member is always disposed obliquely relative to a bottom portion of the vessel.

4. A method according to claim 1, wherein a stirring rod of the stirring member is disposed horizontally.

5. A method according to claim 1, wherein the stirring member is rotated, for stirring, for at least one minute with or without changing the direction of rotation.

6. A method according to claim 1, wherein the stirring member is rotated, for stirring, for a maximum of 10 minutes with or without changing the direction of rotation.

7. A method according to claim 1, wherein, subsequent to the disruption, a buffer solution with the disrupted cells contained therein is drawn out via a pipe which opens into the vessel optionally at a bottom portion of the vessel.

8. A method according to claim 1, wherein the stirring member is driven contactlessly with respect to the vessel.

9. A method according to claim 1, wherein the solution additionally contains enzymes for cell disruption.

10. A device for disrupting cells, comprising a vessel, with beads or particles located in the vessel and/or with a pipe for drawing out liquid from the vessel or feeding liquid into the vessel, and further comprising a stirrer that is optionally magnetic for disrupting cells located in the vessel.

11. A device according to claim 10, wherein the length of a stirring rod of the stirrer is longer than the diameter of a bottom portion of the vessel.

12. A device according to claim 10, comprising a filter or a frit for separating the beads or particles from a liquid with cells contained therein, which is located in the vessel.

13. A device according to claim 12, wherein the frit forms a bottom of the vessel and a pipe for drawing out liquid from the vessel or feeding liquid into the vessel is disposed underneath the frit.

14. A device according to claim 12, wherein the frit has open pores with a diameter of maximally 200 μm.

15. A device according to claim 10, wherein the diameter of the beads or particles is at least 100 μm and/or not more than 1000 μm.

16. A device according to claim 10, wherein the vessel is conical and/or the diameter of the vessel increases towards a top portion thereof.

17. A device according to claim 10, wherein the vessel comprises a cap for sealing the vessel.

18. A device according to claim 10, wherein the vessel is mounted on a substrate and a pipe for drawing out liquid from the vessel or feeding liquid into the vessel is provided in the substrate.

19. A device according to claim 18, wherein the substrate has a funnel-shaped recess underneath a bottom portion of the vessel.

20. A device according to claim 10, wherein cells of bacteria, fungi, animals and/or plants are in the vessel.
